(19) United States
(12) Patent Application Publication
(10) Pub. No.: US 2007/0239041 A1
(43) Pub. Date: Oct. 11, 2007

(54) Non-invasive Venous Pressure Measurement

(75) Inventors: Aniruddha Chatterjee, Baltimore, MD (US); Vikram Aggarwal, Baltimore, MD (US); Yoonju Cho, Baltimore, MD (US); Dickson Cheung, Loan Tree, CO (US)

Correspondence Address: PATENTBEST, 4600 ADELINE ST., #101, EMERYVILLE, CA, 94608, US

(73) Assignee: The Johns Hopkins University, Baltimore, MD (US)

(21) Appl. No.: 11/692,150

(22) Filed: Mar. 27, 2007

Related U.S. Application Data
(60) Provisional application No. 60/787,065, filed on Mar. 28, 2006.

(51) Int. Cl. A61B 5/02; A61B 8/00

(52) U.S. Cl. 600/490; 600/437; 600/453

(57) ABSTRACT

Apparatus and methods for non-invasive measurement of a subject's venous pressure, and particularly the subject's central venous pressure (CVP). The apparatus comprises a probe with a load cell. The method makes use of a non-invasive medical device or technique, such as an ultrasound system, to visualize the internal jugular (IJ) vein. Once the IJ has been located, the operator pushes on the surface of the neck with the probe until the external pressure is sufficient to collapse the IJ. The load cell within the probe determines the amount of force applied, and the applied force is converted into venous pressure.

### CROSS-REFERENCE TO RELATED APPLICATIONS

This application claims priority to U.S. Provisional Patent Application No. 60/787,065, filed on Mar. 29, 2006, the contents of which are incorporated by reference herein in their entirety.

### BACKGROUND OF THE INVENTION

1. Field of the Invention

The invention relates generally to apparatus and methods for measuring venous pressure and, more particularly, to apparatus and methods for non-invasively measuring central venous pressure.

2. Description of Related Art

The central venous pressure (CVP) is an important physiological parameter, the correct measurement of which is a clinically relevant diagnostic tool for heart failure patients, amongst others. For example, increased venous pressure is indicative of low cardiac output and higher blood volume in the venous compartment.

A challenge for physicians is to obtain a quick and accurate measure of a patient's CVP in a manner that poses minimum discomfort. Current methods of measuring CVP accurately are rather invasive. Typically, a catheter is threaded along a major vein until it is within the vicinity of the right atrial compartment. Pressure readings are then collected directly from inside the vein. U.S. Pat. Nos. 6,592,565 and 6,819,951 describe methods and apparatus for collecting CVP data in this manner.

However, threading a central line in this fashion carries certain risks. For example, inserting the needle into the vein itself can result in internal bleeding if an artery is accidentally punctured in the process. The risk of infection is also present whenever the skin is punctured. Furthermore, the procedure is also time-consuming and difficult to perform without hospitalization or in primary care settings.

To avoid performing this procedure unnecessarily, doctors typically first check the CVP non-invasively by treating the superior vena cava as a manometer to the right atrium. The pressure at the right atrium correlates to the height of the column of blood in the vein, which can be estimated by visually identifying small disturbances in the jugular vein. These disturbances are a reflection of the pumping of the atrium and are observed at the topmost part of the column of blood. The actual pressure relative to the heart can be roughly measured by lifting the neck slowly from a supine position until the fluctuations become visible on the surface of the neck. The hydrostatic pressure that corresponds to the height difference between the neck and the heart is a measure of CVP. The procedure is outlined extensively in Lipton, B. “Estimation of central venous pressure by ultrasound of internal jugular vein” American. Journal of Emergency Medicine. 2000 July; 18(4):432-4, the contents of which are incorporated by reference herein in their entirety. This method is prone to error because spotting the exact height where the fluctuations appear is very difficult, especially in patients where layers of fat obscure the jugular vein. The process of physically lifting the patient upward incrementally is also taxing and time-consuming, and not well-suited for emergency conditions.

Other methods have been described to aid physicians in visualizing the exact location of these fluctuations. For example, using ultrasound to visualize the internal jugular vein has been described in the Lipton article cited above. However, in this situation ultrasound only serves to supplant the less accurate visual identification of the fluctuations; lifting the patient to an appropriate height is still required to make an accurate measurement.

Another measurement procedure has been outlined in Baumann U, Marquis C, Stoupis C, Willenberg T A, Takala J, Jakob S M. “Estimation of central venous pressure by ultrasound”. Resuscitation. 64(2005), 193-199, the contents of which are incorporated by reference herein in their entirety. In this study, conducted in Switzerland, the operator uses an ultrasound probe modified with a quartz pressure transducer within a mixture of water and glycerin that is translucent to ultrasound waves. The device records the external pressure needed to collapse the IJ and correlates this value to the CVP. This device requires modification of the ultrasound probe, which makes it unattractive for many clinical care providers, since they would not be able to use their existing ultrasound equipment.

### SUMMARY OF THE INVENTION

One aspect of the invention relates to a venous pressure measurement apparatus. The apparatus comprises a probe, a load cell, and a central unit. The probe has a contacting member constructed and arranged to be pressed against skin proximate to a vein. The load cell is mounted on the probe and coupled to the contacting member such that it is arranged to measure an amount of force exerted by or on the contacting member. The central unit is coupled to the load cell and is adapted to read and record the amount of force measured by the load cell and to convert that measurement to a venous pressure.

Another aspect of the invention relates to a venous pressure measurement system. The system includes a medical device capable of detecting vein closure and a venous pressure measurement apparatus. The apparatus comprises a probe, a load cell, and a central unit. The probe has a contacting member constructed and arranged to be pressed against skin proximate to a vein. The load cell is mounted on the probe and coupled to the contacting member such that it is arranged to measure an amount of force exerted by or on the contacting member. The central unit is coupled to the load cell and is adapted to read and record the amount of force measured by the load cell and to convert that measurement to a venous pressure.

A further aspect of the invention relates to a method of measuring venous pressure. The method comprises visualizing a vein using a non-invasive medical imaging device, applying pressure to the skin over the vein using a probe capable of sensing the applied pressure until the medical device indicates that the vein has begun to collapse, and recording an initial collapse pressure when the vein has begun to collapse. The method also comprises apply pressure to the skin over the vein using the probe until the medical device indicates that the vein has substantially completely closed, recording a final close pressure when the vein has substantially completely closed, and calculating the venous pressure using the difference between the final collapse pressure and the initial collapse pressure.

Other aspects, features, and advantages of the invention will be set forth in the description that follows.

### BRIEF DESCRIPTION OF THE DRAWINGS

The invention will be described with respect to the following drawing figures, in which like numerals represent like features throughout the figures, and in which:

FIG. 1 is a side elevational view of a probe capable of measuring force, according to one embodiment of the invention;

FIG. 2 is a cross-sectional view of the probe of FIG. 1, taken through Line 2-2 of FIG. 1;

FIG. 3 is a top plan view of a central unit adapted to read and record the amount of force measured by the probe of FIG. 1;

FIG. 4 is a schematic view of a system adapted to measure central venous pressure according to another embodiment of the invention; and

FIG. 5 is a schematic flow diagram of a method for measuring central venous pressure using the system of FIG. 4.

### DETAILED DESCRIPTION

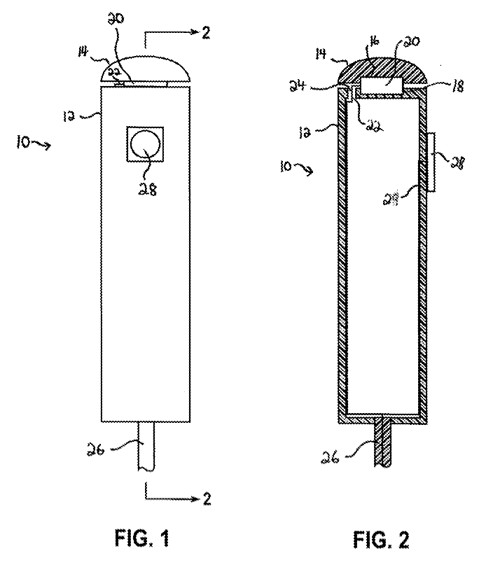

FIG. 1 is a side elevational view of a probe for non-invasively measuring venous pressures, generally indicated at 10. FIG. 2 is a cross-sectional view of the probe 10, taken through Line 2-2 of FIG. 1. The probe 10 has a body 12 and a contacting member 14. The body 12 of the probe 10 in the illustrated embodiment has the form of an elongate, generally cylindrical member with an overall length of approximately 15 cm and a diameter of approximately 2 cm, although in other embodiments, it may have substantially any shape and dimensions, so long as the body 12, or at least a portion thereof, can be held comfortably in a user's hand. In some embodiments, the body 12 may be made of a plastic; in other embodiments, metal may be a suitable material.

The contacting member 14 is constructed and arranged to be pressed against a patient's skin proximate to (e.g., over) a vein to measure the pressure within that vein. In some embodiments, the vein may be the internal jugular (IJ) vein and the pressure measured may be the patient's central venous pressure (CVP).

While the inventors do not wish to be bound by any particular theory of operation, certain aspects of this description may assume that the IJ vein and other veins can be modeled according to Laplace's Law, which is set forth in Equation (1):

$$T = \frac{P \times R}{M} \qquad ( 1 )$$

where T is wall tension, P is transmural pressure, R is vessel radius, and M is wall thickness. According to Laplace's Law, when transmural pressure (the difference between internal and external pressure) is zero, the tension falls to zero and the vessel collapses. Therefore, if the contacting member 14 is pressed against the skin proximate to a vein until the vein collapses, and the pressure exerted to make the vein collapse is measured, the internal pressure of the vein can be determined. In the case of the IJ vein, that determined pressure is the CVP. Specific procedures for determining venous pressures and, in particular, the CVP, will be described below in more detail.

The contacting member 14 of the illustrated embodiment is most advantageously curved, such that when it is pressed against skin, the pressure that it exerts on the skin is concentrated at a single point. (In most applications, that point would be the point along the vein at which pressure is to be measured.) In the embodiment of FIGS. 1-2, the contacting member 14 has an overall semi-hemispherical curvature, although other types of curvature and radii of curvature may be used in other embodiments. The contact area may be on the order of approximately two square centimeters in some embodiments. However, any contact area may be used so long as a proper balance is struck - if the contact area is too small, the contacting member 14 may move past or beyond the vein instead of compressing it; if the contact area is too large, the contacting member 14 may compress soft tissue and other structures as well.

The contacting member 14 may be made of a durable rubber or it may be made of a hard plastic or metal, depending on the embodiment. Generally speaking, it may be advantageous if the contacting member 14 is made of a material that will not deform significantly under the applied loads.

As is shown particularly in the cross-sectional view of FIG. 2, the contacting member 14 is not attached directly to the body 12. Instead, mounted between the contacting member 14 and the body 12, in a recess 16 in the contacting member 14 and a corresponding recess 18 in the top end of the body 12, is a load cell 20. Positioned as shown in FIG. 2, the load cell 20 can perceive all of the forces exerted by or on the contacting member 14. Moreover, so as to ensure accurate measurement, the contacting member 14 does not make direct contact with the body 12 of the probe 10. In some embodiments, however, the contacting member 14 may make contact with other structures, so long as load is not transferred to those structures. For example, the contacting member 14 need not directly contact or cover the load cell 20. Instead, any number of members or elements may be interposed between the load cell 20 and the contacting member 14, so long as those elements are essentially rigid and transmit the full load perceived by the contacting member 14 to the load cell 20 without absorbing or dissipating it.

The precise manner in which the load cell 20 is mounted may vary from embodiment to embodiment. The mounting may be by adhesive, mechanical fastener engagement, or interference fit, depending on the embodiment and the type and capabilities of the load cell 20. In some embodiments, adhesive tape on a flat load cell 20 has been found to be sufficient securement. Adhesives that are flexible when set may also be suitable. In other embodiments, for example, the load cell 20 could include a threaded post on each side, and the contacting member 14 and body 12 could include threaded openings adapted to engage the threads of those threaded posts on the load cell 20. Generally speaking, the contacting member 14 and load cell 20 need not be user-removable or replaceable, although it is advantageous if the contacting member 14 and load cell 20 can be removed and replaced in order to service them.

In one embodiment, the load cell may be, for example, an Omegadyne LCKD-5 five pound subminiature compression load cell (Omegadyne, Inc., Sunbury, Ohio, United States). Load cells of other ranges and sensitivities may be used, so long as they have adequate sensitivity in the range of loads expected for the particular application. Additionally, it should be understood that the term “load cell” is to be construed broadly to include any type of device or element capable of perceiving force and converting that perception into a recordable data point, without regard to the underlying technology by which it does so. For example, piezoelectric load cells, load cells based on change in electrical resistance with deformation, and mechanical spring-deflection load cells may all be used in various embodiments of the invention.

The leads 22 from the load cell 20 pass through a hole 24 in the body 14 bored proximate to the load cell 20, transit the length of the body 14, and exit at the back end of the body 14 in a main data cable 26. Along the interior of the body 14, the leads 22 may be secured to the interior sidewall of the body 14 to reduce the risk of strain and breakage. Additionally, the leads 22 may be covered by a wire guide or another protective structure. In some embodiments, rather than being secured along the interior, the leads 22 may be secured on the exterior of the body 14 and covered by an appropriate protective cover or guide. Ultimately, the manner in which the leads 22 are held within or outside the body is not critical so long as they are not unduly strained and are protected from breakage and other adverse conditions. For that reason, the main data cable 26 may be provided with additional molded strain relief or any other features that may be desirable to protect the leads 22.

On the exterior lateral surface of the body 14, a switching element 28 is provided. In the illustrated embodiment, the switching element 28 is a button, although in other embodiments, the switching element 28 may be a switch or any other sort of element. The leads 29 from the switching element also enter the body 14, traverse its length, and exit in the main data cable 26. As will be explained in greater detail below, when the switching element 28 is actuated, the amount of force measured by the load cell 20 is recorded.

While in operation, the probe 10 may be covered by a disposable cover, such as a disposable latex cover, so as to prevent contamination and avoid transmitting infection from one patient to the next.

In some embodiments, the components used to read and display the load values generated by the load cell 20 and to generate venous pressure values may all be internal to and/or a part of the probe 10, such that the apparatus as a whole comprises only a handheld probe. Ultimately, a unitary probe with all electronics integrated might have a form similar to that of an electronic thermometer, with a display and controls along its exterior sidewall.

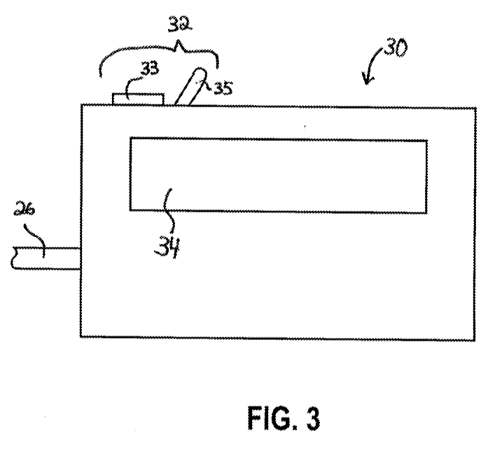

However, in the illustrated embodiment, an external central unit is coupled to the probe 10 through the main data cable 26 in order to read and display the load and pressure values. FIG. 3 is a top plan view of an exemplary external central unit, generally indicated at 30. On its exterior, the central unit 30 has controls 32, including a reset button 33 and a power switch 35, and an external display 34. The type of external display 34 may vary from embodiment to embodiment. For example, the display 34 may be an LED display or an LCD display. Additionally, although configured as a stand-alone unit in the illustrated embodiment, the central unit 30 may be configured to interface and communicate with other medical devices and monitoring tools in other embodiments.

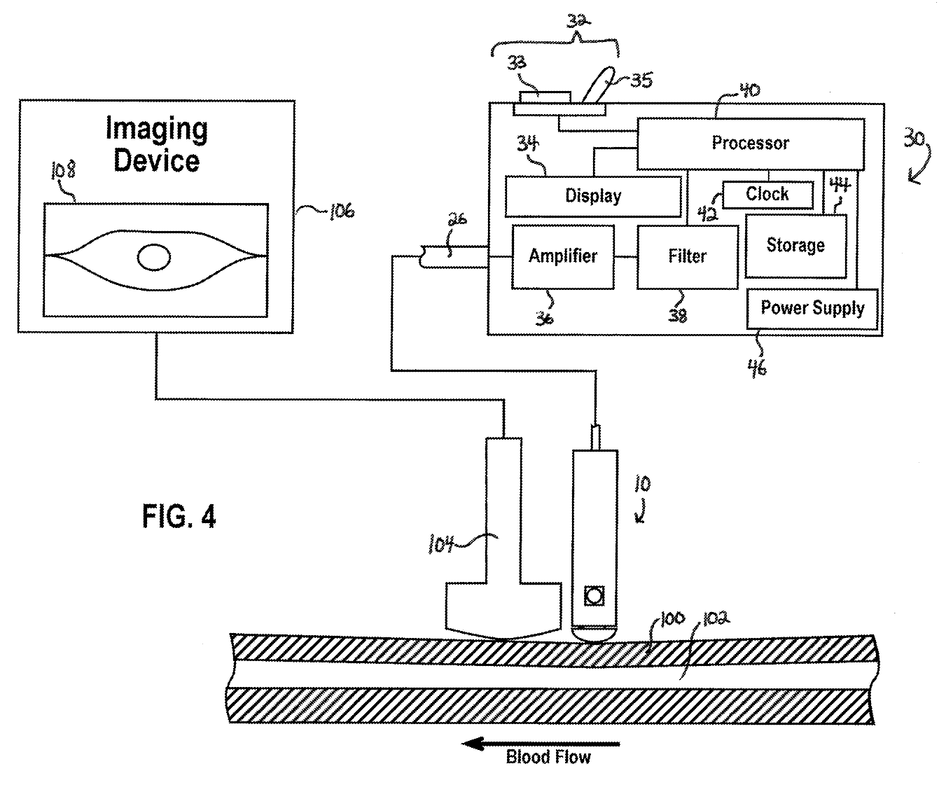

FIG. 4 is a schematic illustration of a system for measuring venous pressure using the probe 10 and central unit 30. FIG. 4 also illustrates the internal components of the central unit 30. As shown, signals from the load cell 20 pass through signal conditioning elements, including an amplifier 36 and a filter 38 which are connected to a processor 40. In other embodiments, other types of signal conditioning elements may be included and interposed between the load cell 20 and the processor 40. Moreover, depending on the type of processor 40, an analog-to-digital converter (ADC) may be used to convert analog voltage signals from the load cell into digital data that can be processed by the processor 40. However, the processor 40 may include an internal ADC.

The processor 40 may be a microcontroller, an ASIC, or any other element capable of performing the described functions. In some embodiments, the central unit 30 may be implemented as a software program on a general purpose computer, in which case the processor 40 may be the CPU of the general purpose computer.

As one example, the amplifier 36 may be an INA128P instrumentation amplifier with a gain of 1,000. The filter 38 may be a low-pass filter based on a LM741 operational amplifier with a cut-off frequency of 0.5 Hz, such that only direct current (DC) signals from the load cell 20 are permitted to pass. The processor may be an 8-bit PIC16F877 microcontroller mounted on an internal circuit board. As shown in FIG. 4, a clock 42, in this case, a 10 MHz crystal oscillator, is coupled to the processor 40, although some processors 40 may include internal clocks, and thus, the clock 42 may be omitted in some embodiments.

The processor 40 may have sufficient onboard storage memory, for example, flash memory, to permit the storage of one or more load readings and/or final pressure readings. However, as shown in FIG. 4, external storage 44 may be provided. The storage 44 may comprise any combination of random access memory (RAM) read-only memory (ROM), programmable read-only memory, and flash memory. Additionally, the storage 44 may include devices that read and write magnetic or optical media, such as hard disk drives, floppy disk drives, CD-ROM drives, CD-R drives, and DVD/DVD-R drives.

The central unit 30 also includes a power supply 46. The power supply 46 may comprise a number of components to allow it to draw power from a number of different sources, including a transformer and AC-to-DC converter to draw power from standard household and industrial power grids, a battery, a rechargeable battery, such as a lithium ion battery, or any combination of those components.

Additionally, as was noted briefly above, the central unit 30 may include one or more input/output ports and their associated hardware in order to communicate with other medical devices, offload venous pressure readings, or otherwise cooperate with other devices. Examples of suitable input/output ports include Universal Serial Bus (USB) ports, IEEE 1394 Firewire ports, RS232-C serial ports, parallel ports, and infrared communication ports. In addition to “wired” input/output ports, some embodiments of the invention may also be equipped for wireless communication, such as by the 802.11a/b/g and Bluetooth wireless networking standards, or by wireless standards and hardware specific to medical devices.

In order to measure a venous pressure value, the probe 10 and its central unit are used in combination with a technology that allows the user to determine when the vein in question has begun to collapse and when it has substantially completely closed. A number of different technologies, particularly medical imaging technologies, may be used. For example, ultrasound systems are suitable, as are Doppler imaging systems. However, other technologies may be used, such as auscultation for characteristic noises indicating vein closure and other auditory sensing techniques.

Certain aspects of the following description may assume the use of ultrasound, which is presently one of the most commonly available types of medical imaging technologies suitable for the purpose. However, the particular type of technology or technique used to determine when the vein has begun to collapse and when it has substantially completely collapsed is not critical to the invention, so long as the technology or technique is appropriately calibrated and verified to function correctly.

As shown in FIG. 4, in order to measure venous pressure, the probe 10 is placed in contact with the skin 100 over the vein 102 in which venous pressure is to be measured. If the venous pressure to be measured is a CVP, then the vein 102 would generally be the IJ vein.

Placed proximate to the probe 10 is the probe 104 of an imaging device 106. The placement of the probe 104 of the imaging device 106 relative to that of the probe 10 may vary from embodiment to embodiment and from one application or patient to another. In some embodiments, the probe 104 of the imaging device 106 may be placed closer to the patient's heart than the probe 10, because it may be easier to visualize the collapsing vein from that vantage point. However, in other embodiments, the probe 10 may be placed closer to the heart than the probe 104 of the imaging device 106. Other factors may also come into play to determine the placement of the probes 10, 104 relative to one another. For example, it may be necessary or desirable to choose the placement of one relative to another so that there is a suitable muscular backing onto which to push the vein so that it will collapse under the exerted force. Ultimately, it is generally advantageous if the two probes 10, 104 are placed close to one another, for example, within about one inch of one another.

Using the display 108 of the imaging device 106, the user is able to visualize the changes in the vein 102 as pressure is exerted by the probe 10. When the vein 102 begins to collapse, the user actuates the switching element 28 on the probe 10 to store that force value; when the display 108 of the imaging device 106 indicates that the vein 102 has substantially completely closed, the user actuates the switching element 28 again to store that final force value and calculate the venous pressure. Generally speaking, the difference between the initial and final force values is taken to be the venous pressure, although, as will be described below, that value may be transformed or modified to account for calibration or other issues. In the view of FIG. 4, the vein 102 is slightly compressed where the probe 10 contacts it and has thus begun to collapse.

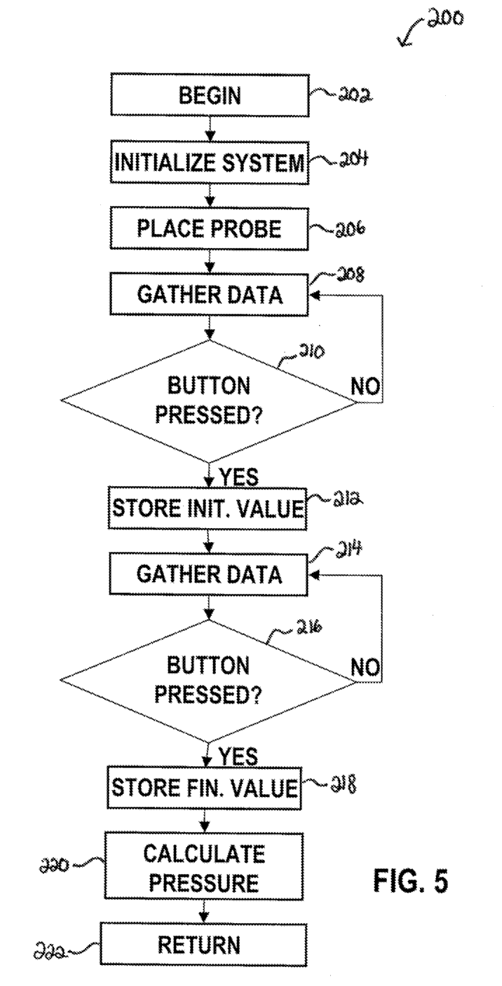

More specifically, this process is described in the flow diagram of FIG. 5, which illustrates a method 200 for determining a venous pressure. Method 200 begins at task 202 and continues with task 204. In task 204, the system is initialized. Initialization may include a number of steps. For example, an initial reading may be taken from the load cell 20 and that reading may be used to zero the load cell 20. Additionally, if a calibration curve for the load cell or other calibration data is available, that data may be retrieved during the initialization. In some embodiments, task 204 may also involve initializing components internal to the central unit 30 or the processor 40, such as the analog-to-digital converter,

Method 200 continues with task 206, in which the user places the probe 10 over the vein 102, as illustrated in FIG. 4. Once the user has placed the probe 10, the central unit 30 essentially executes a loop until the switching element 28 is actuated. Specifically, in task 208, a load data point is gathered from the load cell 20. Method 200 then continues with task 210, in which it is determined whether the switching element 28 has been actuated. If the switching element 28 has been actuated (task 210:YES), indicating initial vein collapse, then method 200 continues with task 212 and the data point gathered in task 208 is stored as the force value at initial vein collapse. If the switching element 28 has not been actuated, then method 200 returns to task 208 and another data point is gathered.

Once the initial force value, indicating the beginning of vein collapse, is stored in task 212, another data point is gathered in task 214. After a data point is gathered in task 214, method 200 continues with task 216, another decision task in which it is determined whether the switching element 28 has been actuated to indicate that the vein has substantially completely closed. If the switching element has been actuated (task 216:YES), method 218 continues with task 218 and data point gathered in task 216 is stored as the final pressure at vein closure. If the switching element has not been actuated (task 216:NO) method 200 returns to task 214.

After task 218, method 200 continues with task 220, in which the venous pressure is calculated. In some embodiments, the venous pressure may be calculated as the simple difference between the force applied to cause final vein closure and the force applied to cause initial vein collapse. For purposes of description, the venous pressure established by taking the simple difference between the final and initial applied forces will be referred to as the simple difference pressure.

As those of skill in the art will realize, it may be necessary to transform the simple difference pressure using linear or nonlinear functions to account for a number of conditions or factors so as to arrive at a precise, accurate final venous pressure reading. For example, it may be advantageous to calibrate the load cell 20 by measuring its response to a series of known weights or pressures and then transforming the simple difference pressure using the calibration data. A number of techniques for calibrating load cells are known in the art and any may be used.

Additionally, as those of skill in the art will realize, venous pressures measured with this technique may vary with the characteristics of the individual patients, including the patient's age, gender, and other characteristics. Therefore, it may be useful to calibrate the measurement technique itself to establish calibration data. For example, a probe 10, and the technique for using it, could be calibrated by performing method 200 on a patient, simultaneously performing a typical venous catheterization to measure venous pressure internally, and comparing the data obtained by the two results.

If the simple difference pressure is to be transformed, then task 204, in which the system is initialized, could also comprise retrieving the appropriate transformation factors or functions, or, in some embodiments, allowing the user to select which of a plurality of transformation factors should be used. This could be done, for example, by allowing the user to specify the age, gender, and other characteristics of the patient.

Although not shown in FIG. 5, once it is gathered, a pressure measurement may optionally be stored in the storage 44 so that it can be reviewed at a later point.

In pseudocode, not specific to any particular computer or machine programming language, the tasks of method 200 may be rendered as:

```
main( )
  // start-up waiting period - flash 7-Seg Display while resetting
  for (i = 0; i < 2; i++)
    display(ZERO)
    delay
    display(OFF)
    delay
  end for
  // setup A/D converter
  setup_adc( );
  // calculate initial tension on load cell
  init_calib_value = read_adc( );
  // set initial tension to part of initial skin compliance
  init_skin_value = init_calib_value;
  // program loops continuously until reset
  while (TRUE)
    if not final value then
      // keep reading data
      ADC_result = read_adc( ) − init_skin_value
    end if
    // convert ADC value (0-255) to CVP value (0-20)
    CVP = convert(ADC_result)
    if valid CVP then
      display(CVP)
    else
      display(ZERO)
    end if
    if button pushed then
      if button pushed for first time then
        // zero out pressure reading to account for initial tension
        init_skin_value = init_skin_value + ADC_result
        set indicator LED
      else if button pushed for second time then
        // record final value and stop further reading
        set indicator LED
      end if
    end if
  end while
end main( )
```

The above pseudocode assumes that the display 34 is a seven-segment LED display. Additionally, the abbreviation “ADC” refers to the analog-to-digital converter of the processor 40.

Although the invention has been described with respect to certain exemplary embodiments, the examples are intended to be illuminating, rather than limiting. Modifications and changes may be made within the scope of the invention, which is determined by the claims.

What is claimed is:

1. A venous pressure measurement apparatus, comprising:
a probe having a contacting member constructed and arranged to be pressed against skin proximate to a vein;
a load cell mounted on the probe and coupled to the contacting member, the load cell being arranged to measure an amount of force exerted by or on the contacting member; and
a central unit coupled to the load cell, the central unit being adapted to read and record the amount of force measured by the load cell and to convert that measurement to a venous pressure.

2. The venous pressure measurement apparatus of claim 1, further comprising a switching element coupled to the central unit, the switching element being constructed and arranged such that, when activated, the amount of force measured by the load cell is recorded by the central unit.

3. The venous pressure measurement apparatus of claim 2, wherein the switching element is provided on an exterior surface of the probe.

4. The venous pressure measurement apparatus of claim 1, wherein the contacting member is connected to the probe through the load cell, such that the amount of force exerted by or on the contacting member is measured directly by the load cell.

5. The venous pressure measurement apparatus of claim 1, further comprising one or more signal conditioning elements selected from the group consisting of a filter and an amplifier coupled between the load cell and the central unit.

6. The venous pressure measurement apparatus of claim 1, wherein the contacting member is detachable from the probe.

7. The venous pressure measurement apparatus of claim 6, wherein the contacting member is comprised of rubber.

8. The venous pressure measurement apparatus of claim 1, wherein the venous pressure is a central venous pressure.

9. A venous pressure measurement system, comprising:
a medical device constructed and arranged to detect the closure of a vein; and
a venous pressure measurement apparatus, the apparatus including:
a probe having a contacting member constructed and arranged to be pressed against skin proximate to a vein,
a load cell mounted on the probe and coupled to the contacting member, the load cell being arranged to measure an amount of force exerted by or on the contacting member, and
a central unit coupled to the load cell, the central unit being adapted to read and record the amount of force measured by the load cell and to convert that measurement to a venous pressure.

10. The venous pressure measurement system of claim 9, the venous pressure measurement apparatus further comprising a switching element coupled to the central unit, the switching element being constructed and arranged such that, when activated, the amount of force measured by the load cell is recorded by the central unit.

11. The venous pressure measurement system of claim 9, wherein the switching element is provided on an exterior surface of the probe.

12. The venous pressure measurement apparatus of claim 9, wherein the contacting member is connected to the probe through the load cell, such that the amount of force exerted by or on the contacting member is measured directly by the load cell.

13. The venous pressure measurement apparatus of claim 9, the venous pressure measurement apparatus further comprising one or more signal conditioning elements selected from the group consisting of a filter and an amplifier coupled between the load cell and the central unit.

14. The venous pressure measurement apparatus of claim 9, wherein the medical device comprises an ultrasound system.

15. The venous pressure measurement apparatus of claim 9, wherein the medical device comprises a Doppler system.

16. A method of measuring a venous pressure, comprising:
visualizing a vein using a non-invasive medical imaging device;
applying pressure to the skin over the vein using a probe capable of sensing the applied pressure until the medical imaging device indicates that the vein has begun to collapse;
recording an initial collapse pressure when the vein has begun to collapse;
applying pressure to the skin over the vein using the probe device until the medical imaging device indicates that the vein has substantially completely closed;
recording a final close pressure when the vein has substantially completely closed; and
calculating the venous pressure using the difference between the final close pressure and the initial collapse pressure.

17. The method of claim 16, wherein the medical imaging device is an ultrasound system.

18. The method of claim 16, wherein the pressure is the central venous pressure.

19. The method of claim 18, wherein the vein is the internal jugular vein.
